(19) United States
(12) Patent Application Publication
(10) Pub. No.: US 2005/0244807 A1
(43) Pub. Date: Nov. 3, 2005

(54) Two-photon absorption heteroaromatic chromophores and compositions thereof

(75) Inventors: Alessandro Abbotto, Milano (IT); Luca Beverina, Milano (IT); Renato Bozio, Selvazzano Dentro (IT); Giorgio A. Pagani, Milano (IT); Raffaella Signorini, Noventa Padovana (IT)

Correspondence Address: Gifford Krass Groh Sprinkle Anderson & Citkowski, P O Box 7021, Troy, MI, 48007-7021, US

(73) Assignee: UNIVERSTA' DEGLI STUDI DI MILANO-BICOCCA, Milan (IT)

(21) Appl. No.: 10/523,751

(22) PCT Filed: Jul. 8, 2003
(86) PCT No.: PCT/EP03/07300

§ 371 Date: Feb. 9, 2005

(30) Foreign Application Priority Data
Aug. 9, 2002 (IT) MI2002A001809

(51) Int. Cl. C07D 49/14; C07D 45/14

(52) U.S. Cl. 435/4; 546/256; 546/276.1; 546/281.1

(57) ABSTRACT

The present invention relates to new heteroaromatic compounds with high two-photon absorption activity, useful in particular as optical power limiting agents via two-photon absorption or as imaging agents in confocal laser scanning fluorescence microsopy via two-photon absorption or excitation.

The present invention relates to new heteroaromatic chromophores with significant two-photon absorption activity.

It is known that molecules exhibit a nonlinear optical (NLO) behaviour by simultaneously absorbing two or more photons, either of the same or of different energy, to be promoted to one of their excited states when exposed to an intense laser pulse. In the case of two-photon absorption (TPA), the frequency of the nonlinear absorption is approximately half of that corresponding to the conventional linear one-photon absorption. As a consequence, the TPA frequency typically falls in the visible red-near infrared (NIR) region of the electromagnetic radiation spectrum, where the material is transparent with respect to one-photon absorption. TPA is a 3rd order NLO process and is described by the imaginary part of the 3rd order nonlinear susceptibility.

Once the molecule has reached one of its excited states via TPA, it may show a fluorescence emission to return to its ground-state. In particular, the two-photon induced fluorescence emission occurs at a frequency very similar to the one-photon induced fluorescence emission. The direct consequence of this phenomenon is that the two-photon excited fluorescence frequency is usually larger than the TPA frequency, as opposed to the case of linear absorption, where the emitted frequency is always smaller than that absorbed. Therefore, TPA dyes may absorb a red or NIR radiation (low frequency) and emit in the visible range.

Organic molecules able to show a significant TPA activity are very important for a variety of emerging applications including optical limiting (eye and sensor protection), three-dimensional optical memories, two-photon laser scanning fluorescence microscopy, up-converted lasing, non-destructive imaging of coated materials, and micro- and nanofabrication (MEMS, microelectromechanical systems) (Denk, W.; Strickler, J. H.; Webb, W. W. Science 1990, 248, 73; Ehrlich, J. E.; Wu, X. L.; Lee, L. Y. S.; Hu, Z. Y.; Rockel, H.; Marder, S. R.; Perry, J. W. Opt. Lett. 1997, 22, 1843; Day, D.; Gu, M.; Smallridge, A. Opt Lett. 1999, 24, 948; Cumpston, B. H.; Ananthavel, S. P.; Barlow, S.; Dyer, D. L.; Ehrlich, J. E.; Erskine, L. L.; Heikal, A. A.; Kuebler, S. M.; Lee, I. Y. S.; McCord-Maughon, D.; Qin, J. Q.; Rockel, H.; Rumi, M.; Wu, X. L.; Marder, S. R.; Perry, J. W. Nature 1999, 398, 51; Belfield, K. D.; Ren, X. B.; Van Stryland, E. W.; Hagan, D. J.; Dubikovsky, V.; Miesak, E. J. J. Am. Chem. Soc. 2000, 122, 1217; Abbotto, A.; Beverina, L.; Bozio, R.; Bradamante, S.; Pagani, G. A.; Signorini, R. Synth. Met. 2001, 121, 1755; Abbotto, A.; Beverina, L.; Bozio, R.; Bradamante, S.; Ferrante, C.; Pagani, G. A.; Signorini, R. Adv. Mater. 2000, 12, 1963).

The nonlinear absorption provides many advantages with respect to the conventional technologies based on linear absorption: a) two-photon excitation occurs in the red or NIR region; this region overlaps with the optical transparency window of cells and living tissues; as a consequence, TPA provides much deeper light penetration depths as opposed to conventional techniques; b) the absorbed TPA intensity scales quadratically with the intensity I of the incident laser radiation, which in turn decreases approximately as the square of the distance from the focus; the consequence is that molecules are excited via TPA only at the focus of the beam; two-photon induced phenomena occur only at the focus as well, with a fourth power increased spatial resolution; c) red and NIR light scattering is minimized with respect to higher frequency radiation.

The TPA phenomenon has been theoretically predicted by Göppert-Mayer in 1931 (Göppert-Mayer, M. Ann. Phys. 1931, 9, 273) and experimentally confirmed 30 years later (Kaiser, W. K.; Garrett, C. G. B. Phys. Rev. Lett. 1961, 7, 229). However, TPA has been studied in more detail only with the availability of proper laser sources. Moreover, all of TPA based applications remained unexplored for decades due to the lack of efficient TPA absorbers. Only recently a number of dyes exhibiting significant TPA activity have been proposed. The vast majority of these molecules are based on benzenoid derivatives substituted with conventional donor and acceptor groups such as NO₂, CN, SOₙR, CO₂R, OR e NR₂.

Few examples of TPA chromophores based on substituted heteroaromatic compounds are known. Concerning this aspect, WO 01/70735 owned by the same Applicant is mentioned.

In accordance with the present invention, new molecules are provided with high TPA activity, via excitation with lasers operating in the visible-red or NIR wavelength region, that is a range where most organic molecular and polymeric materials and organic tissues are highly transparent.

In accordance with the present invention, compounds are provided having the following general formulas (I) and (II) 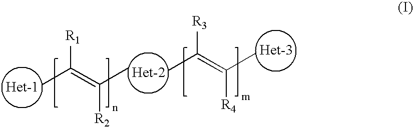
wherein Het-1 and Het-3 are identical or different, and are selected among the following heterocyclic groups: 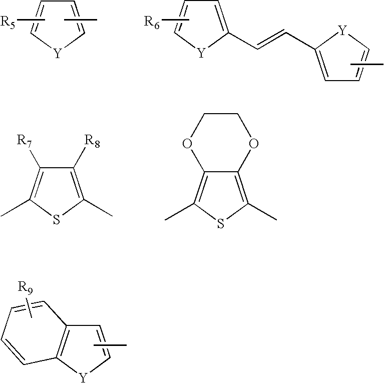
wherein Y may be O, S, or NZ with Z=H, lower alkyl, and aryl; and wherein R₅, R₆, R₇, R₈, and R₉ are the same or different, and are selected from the group consisting of H, alkyl groups having from 1 to 18 carbon atoms, alkoxy, aminoalkyl, alkylhalide, hydroxyalkyl, alkyl groups containing hydroxy and amino functionalities, alkoxyalkyl, alkylsulfide, alkylthiol, alkylazide, alkylcarboxylic, alkylsulfonic, alkyylisocyanate, alkylisothiocyanate, alkylalkene, alkylalkyne, aryl, formyl, and that can contain electronpoor ethenylic moieties such as maleimide, capable to react with nucleophilic groups such as —SH, and groups such as isothiocyanate capable to react with groups such as —NH₂;
and Het-2 is selected among the following heterocyclic groups: 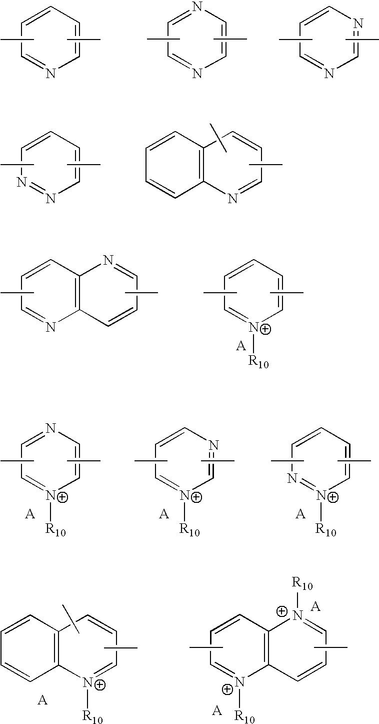
wherein R₁₀ is selected from the group consisting of H, alkyl groups having from 1 to 18 carbon atoms, alkoxy, aminoalkyl, alkylhalide, hydroxyalkyl, alkyl groups containing hydroxy and amino functionalities, alkoxyalkyl, alkylsulfide, alkylthiol, alkylazide, alkylcarboxylic, alkylsulfonic, alkyylisocyanate, alkylisothiocyanate, alkylalkene, alkylalkyne, aryl, formyl, and that can contain electronpoor ethenylic moieties such as maleimide, capable to react with nucleophilic groups such as —SH, and groups such as isothiocyanate capable to react with groups such as —NH₂;
and A is selected among the anions alkylsulfonate, arylsulfonate, polyarenesulfonate, triflate, halide, sulfate, methosulfate, phosphate, polyphosphate,
and wherein n and m, the same or different may be 0, 1, 2;
and R₁, R₂, R₃, and R₄, the same or different, may be H, lower alkyl, alkoxyalkyl, aryl, cyano, alkoxycarbonyl, —(CR₁₁R₁₂)ₚ-Het, wherein 0<p<10, R₁₁ and R₁₂, the same or different, are selected from the group of H, lower alkyl, and Het may be Het-1 or Het-2 or Het-3. 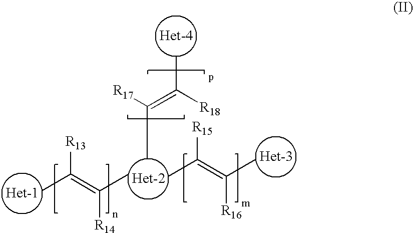
wherein Het-1, Het-3, and Het-4 are the same or different and are selected among the following heterocyclic groups: 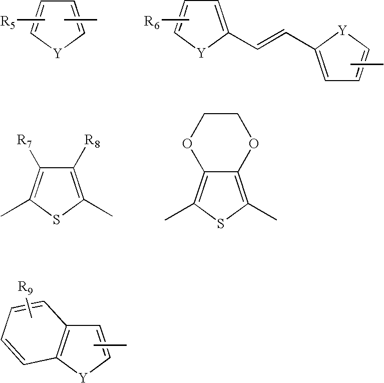
wherein Y may be O, S, and NZ with Z=H, lower alkyl, aryl;
and R₅ and R₆, are the same or different, and are selected from the group consisting of H, alkyl groups having from 1 to 18 carbon atoms, alkoxy, aminoalkyl, alkylhalide, hydroxyalkyl, alkyl groups containing hydroxy and amino functionalities, alkoxyalkyl, alkylsulfide, alkylthiol, alkylazide, alkylcarboxylic, alkylsulfonic, alkyylisocyanate, alkylisothiocyanate, alkylalkene, alkylalkyne, aryl, formyl, ketone, and that can contain electronpoor ethenylic moieties such as maleimide, capable to react with nucleophilic groups such as —SH, and groups such as isothiocyanate capable to react with groups such as —NH₂; R₅, and R₆, the same or different, may further be the following heterocyclic group: 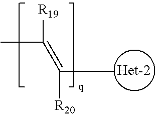
and R₇, R₈, and R₉ are defined as above;
and Het-2 is defined as above;
and wherein n, m, p, and q, the same or different, may be 0, 1, or 2;
and wherein R₁₃, R₁₄, R₁₅, R₁₆, R₁₇, R₁₈, R₁₉, and R₂₀ are the same or different and are selected from the group of H, lower alkyl, alkoxyalkyl, aryl, cyano, alkoxycarbonyl, —(CR₂₁R₂₂)ₗ-Het, wherein 0<l<10, and R₂₁ e R₂₂, the same or different, are selected from the group of H, lower alkyl, and Het may be Het-1 or Het-2 or Het-3, or Het-4.

For the uses according to the present invention said compounds can show their two-photon absorption activity as such, or once prepared in solution, or in a solid state.

In a further aspect of the present invention said compounds can be processed into compositions containing a polymer material such as poly(methacrylate), polyimide, polyamic acid, polystyrene, polycarbonate, and polyurethane or an organically-modified silica (SiO₂) network.

In particular, in said compositions the said compounds are either dispersed or covalently bonded to the polymer materials or to the silica network.

Features and advantages of the present invention will become readily apparent by reference to the following detailed description in conjunction with the accompanying drawings, in which:

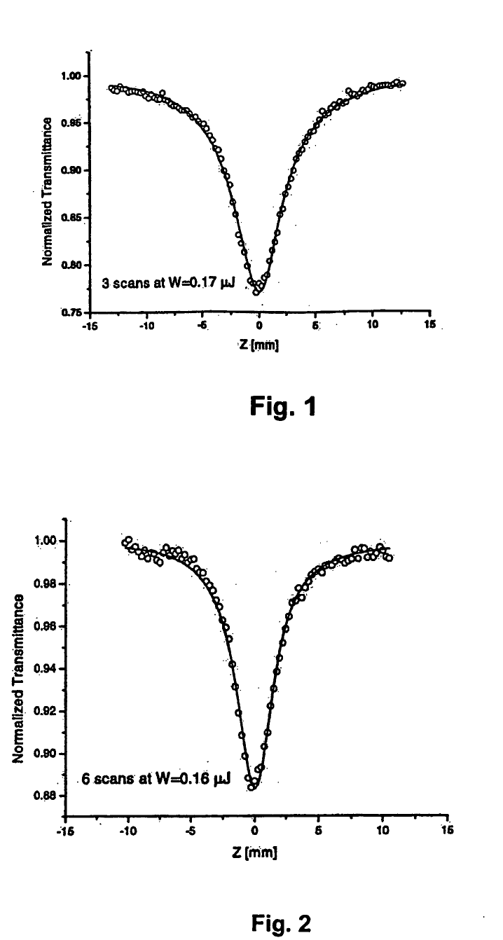

FIG. 1 shows a typical two-photon absorption profile of compound (3) in DMSO (dimethylsulfoxide) obtained via the open-aperture Z-scan technique;

FIG. 2 shows a typical two-photon absorption profile of compound (6) in DMSO obtained with the same technique.

A detailed description of the invention is provided, with reference to certain compounds, which possess a structure corresponding to the formulas (3), (6), and (7), with examples which are not limiting the present invention.

### EXAMPLE 1

Compound (3), endowed with TPA properties, has been prepared by a triple condensation reaction of compound (1) (Zhu, D.; Kochi, Jay K. Organometallics 1999, 18, 161) with an excess of N-methyl-2-pyrrolecarboxaldehyde in refluxing n-butanol in the presence of a catalytic amount of piperidine as a base.

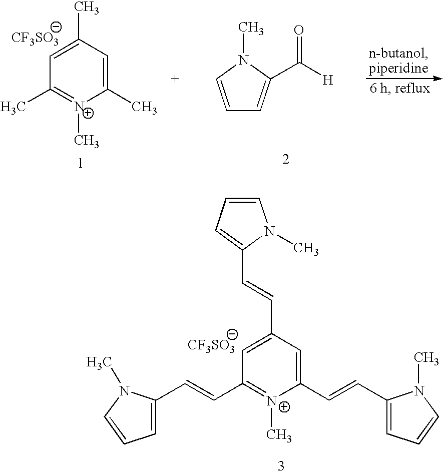

N-methyl-2,4,6-[1-(N-methylpyrrol-2-yl)ethen-2-yl]pyridinium triflate (3). A solution of N-methyl-2-pyrrolecarboxaldehyde (1.545 g, 14.16 mmol) in n-butanol (10 mL) was added dropwise to a solution of N-methyl-sym-collidinium triflate (1.332 g, 4.42 mmol) in the same solvent (40 mL). Ten drops of piperidine were added to the colorless solution and the mixture was stirred at reflux for 6 h. The resulting red-violet mixture was concentrated to ca. 15 mL and the red precipitate collected under reduced pressure. The solid was washed with toluene (10 mL) to give the product (1.783 g, 3.21 mmol, 68%) mp 250° C. (dec) (n-BuOH); ¹H-NMR (CDCl₃) δ 7.78 (2 H, s), 7.63 (1 H, d, J=16.0), 7.52 (2 H, d, J=15.4), 6.90 (1 H, d, J=16.0), 6.82 (1 H, d, J=3.8), 6.78 (2 H, s), 6.75 (2 H, d, J=15.4), 6.75 (2 H, d, J=3.7), 6.72 (1 H, s), 6.21 (2 H, d, J=3.2), 6.17 (1 H, d, J=3.2), 3.95 (3 H, s), 3.83 (6 H, s), 3.82 (3 H, s); EA calcd for C₂₈H₂₉F₃N₄O₃S: C, 60.20%; H, 5.23%; N, 10.03%. Found: C, 60.60%; H, 5.29%; N, 9.62%.

We describe now a non limitative example related to a compound of general formula (I) and defined by the formula (6).

### EXAMPLE2

Compound (6) has been prepared by a condensation reaction of aldehyde (5) (Akoudad, S.; Frere, P.; Mercier, N.; Roncali, J. J. Org. Chem. 1999, 644267) and pyridinium salt (4) (Zhu, D.; Kochi, J. K. Organometallics 1999, 18, 161) in refluxing ethanol and in the presence of a catalytic amount of piperidine.

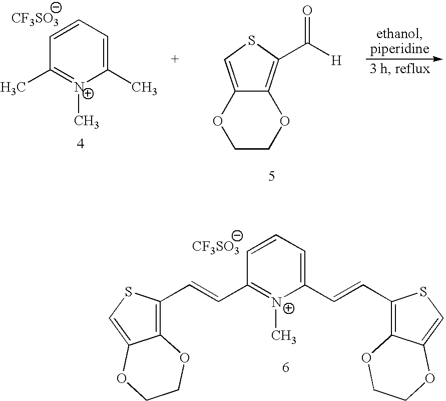

N-methyl-2,6-[1-(3,4-ethilenedioxythiphen-2-yl)ethen-2-yl]pyridinium triflate (6). A solution of 3,4-ethylenedioxythiophene-2-carboxaldehyde (0.456 g, 2.7 mmol) in ethanol (10 ml) was added dropwise to a solution of 1,2,6-trimethylpyridinium triflate (0.350 g, 1.3 mmol) and a few drops of piperidine in the same solvent (20 ml). Reaction mixture was refluxed for 3 hours and then cooled to 0° C. giving the formation of a brown-yellow precipitate that was filtered under reduced pressure and crystallized from ethanol (0.539 g, 0.94 mmol, 72%). mp 103-105° C. ¹H-NMR (DMSO-d₆) δ 8.24 (1 H, t, J=8.14), 8.16 (2 H, d, J=8.09), 7.66 (2 H, d, J=15.54), 7.11 (2 H, d, J=15.63), 6.97 (2 H, s), 4.40 (4 H, m), 4.29 (4 H, m), 4.11 (3 H, s); ¹³C-NMR (DMSO-d₆) 153.47 (2 C), 143.41 (2 C), 142.28 (1 C), 142.02 (2 C), 131.50 (2 C), 126.40 (2 C), 122.64 (2 C), 114.16 (2 C), 104.56 (2 C), 65.27 (2 C), 64.30 (2 C), 41.16 (2 C). Anal Calcd. for C₂₃H₂₀F₃NO₇S₃: C, 47.99%; H, 3.50%; N, 2.43%. Found: C, 47.90%; H, 3.11%; N, 2.20%.

We describe now another non limitative example related to a compound of general formula (II) and defined by the formula (7).

### EXAMPLE 3

Compound (7) has been prepared following a two-step procedure. In the first step the sim-collidinium salt (8) has been condensed with an excess of aldehyde (9) (Abbotto, A.; Beverina, L.; Bozio, R.; Facchetti, A.; Ferrante, C.; Pagani, G. A.; Pedron, D.; Signorini, R. Org. Lett., 2002, 4, 1495) in hot propylene glycol and in the presence of catalytic piperidine. Alkylation of the crude reaction product with an excess of cetyltriflate (Abbotto, A.; Bradamante, S.; Facchetti, A.; Pagani, G. A. J. Org. Chem. 1997, 62, 5755) in anhydrous acetonitrile gave the pure title compound.

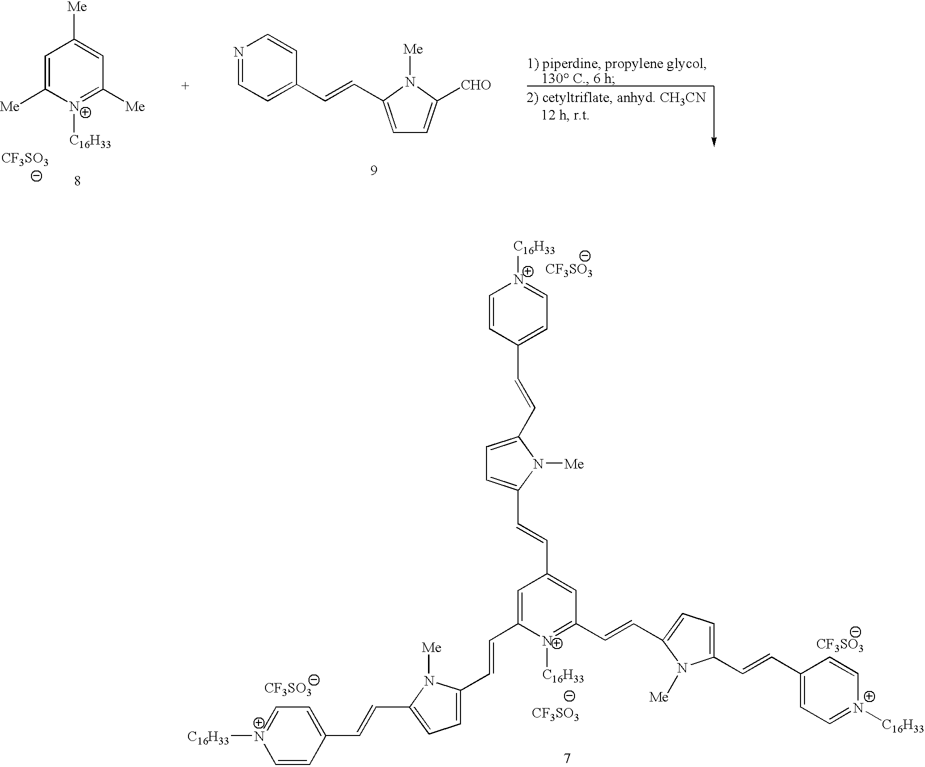

N-cetyl-2,4,6-trimethylpyridinium triflate (8). A solution of cetyl triflate (1.873 g, 5 mmol) in dry toluene (5 ml) was added to a solution of sym-collidine (0.606 g, 5 mmol) under dry atmosphere. The white solution was heated at about 60° C. for 1 hour and then solvent was evaporated. The white residue was taken up with diethyl ether (10 ml) and filtered under reduced pressure, yielding the product as a white solid (1.982 g, 4 mmol, 80.0%) mp 54-56° C.

N-cetyl-2,4,6-[1-[N-methyl-5-(1-(pyrid-4-yl)-ethen-2-yl)pyrrol-2-yl]ethen-2-yl]pyridinium triflate (7). A solution of (9) (1.306 g, 6.1 mmol) in propylene glycol (15 mL) was added to a solution of N-cetyl-sym-collidinium triflate (0.460 mg, 0.93 mmol) and piperidine (5 drops) in the same solvent (10 mL). The resulting orange mixture was heated at 130° C. for 6 h yielding a dark violet solution which was cooled to room temperature and poured into Et₂O (100 mL). The obtained precipitate was collected by filtration under reduced pressure to give the monoquaternized precursor of (7) as a black solid, which was washed with water (20 mL) and EtOH (5 mL) (0.270 mg, 0.25 mmol, 27%) mp>350° C. (dec); ¹H NMR (DMSO-d₆) δ 8.53 (6 H, d, J=4.6), 8.23 (2 H, s), 7.99 (1 H, d, J=15.7), 7.75 (2 H, d, J=15.0), 7.62 (2 H, d, J=16.2), 7.61 (1 H, d, J=15.0), 7.60 (1 H, d, J=14.8), 7.58 (6 H, d, J=4.2), 7.26 (2 H, d, J=15.2), 7.17 (2 H, d, J=3.7), 7.10 (3 H, d, J=15.9), 6.99 (1 H, d, J=4.2), 6.91 (3 H, m), 4.60 (2 H, t, broad), 3.96 (3 H, s), 3.95 (6 H, s), 1.78 (2 H, m, broad), 1.10-1.50 (26 H, m), 0.82 (3 H, t, J=6.7); ¹³C NMR (DMSO-d₆) d 151.60 (2 C), 149.94 (6 C), 149.93 (1 C), 144.43 (3 C), 136.22 (1 C), 135.99 (2 C), 133.38 (1 C), 133.07 (2 C), 129.24 (2 C), 127.33 (1 C), 125.32 (1 C), 125.17 (2 C), 121.09 (3 C), 120.54 (6 C), 119.68 (1 C), 117.79 (2 C), 114.13 (2 C), 112.89 (2 C), 112.22 (1 C), 110.65 (1 C), 110.21 (2 C), 49.95 (1 C), 31.26 (1 C), 30.83 (1 C), 30 73 (2 C), 28.50-29.20 (12 C), 28.33 (1 C), 28.23 (1 C), 25.42 (1 C). A solution of cetyl triflate (2.050 g, 5.95 mmol) in anhyd. CH₃CN (40 mL) was added, under nitrogen atmosphere, to a solution of the product obtained as described in the previous step (1.195 g, 1.11 mmol) in the same solvent (80 mL). The reaction mixture was stirred overnight at room temperature and the solvent evaporated to leave a residue which was taken up with Et₂O (30 mL). The dark precipitate was collected by filtration under reduced pressure and washed several times with boiling hexane. The resulting blue solid was treated with boiling water to give the product (1.357 g, 0.62 mmol, 55.9%). mp>350° C. (EtOH); ¹H NMR (DMSO-d₆) δ 8.80 (4 H, m), 8.63 (2 H, m), 8.30 (2 H, s), 8.26 (1 H, d, J=14.1), 8.18-8.15 (4 H, m), 8.02-7.96 (3 H, m) 7.92-7.86 (2 H, m), 7.82-7.74 (3 H, m), 7.39 (1 H, d, J=15.3), 7.31-7.24 (3 H, m), 7.23-7.14 (3 H, m), 7.12-7.09 (2 H, m), 7.05 (1 H, m), 7.01 (1 H, m), 4.68 (2 H, t broad), 4.45 (6 H, t broad), 3.98 (6 H, s), 3.97 (3 H, s), 1.90 (6 H, m broad), 1.77 (2 H, s broad), 1.45-1.10 (104 H, m), 0.90-0.80 (12 H, m).

According to the present invention said compounds show large two-photon absorption cross-sections both in solution and in the solid state. We now describe, with examples which are not limiting the present invention, experimental data of the two-photon absorption activity of said compounds (3), (6), and (7).

We define the following parameters: β (two-photon absorption coefficient, concentration dependent), σ₂ and σ₂′ (cross-sections). It is possible to obtain the absorption coefficient β by interpolation of the relationship between the transmittance T versus the initial laser beam intensity I₀, in accordance with the following relationships:

$$T = \frac{ln ( 1 + {I}_{0} L β )}{{I}_{o} L β} where$$

$$T = \frac{{I}_{t}}{{I}_{o}}$$

and L=1 cm and Iₜ is the intensity of the trasmitted beam. The I₀ and Iₜ dimensions are Iₜ, I₀=[GW/cm²] whereas the β dimensions are β=[cm/GW]

Since

$${σ}_{2} = \frac{{β}^{′}}{{N}_{a}}$$

it follows that

$${σ}_{2} = \frac{β}{{N}_{a} c} {10}^{3}$$

where Nₐ is the Avogadro's number and σ₂ has the dimensions of [cm⁴/GW].

Finally: σ′₂=hvσ₂. σ′₂ has the dimensions of

$$[ \frac{{cm}^{4} \cdot s}{photon \cdot molecule} ] .$$

The following table summarizes the nonlinear optical characterization data for said compounds taken as examples.

| Compound | λ(nm) | Pulse duration (fs) | Power (μJ) | Intensity (GW/cm²) | Concentration (mmoli/l) | β(cm/GW) | $\begin{matrix}{σ}^{′} 2 \\ [ \frac{{10}^{- 50} {cm}^{4} \cdot s}{photon \cdot molecule} ]\end{matrix}$ |
|---|---|---|---|---|---|---|---|
| 3 | 785 | 130-150 | 0.14 | 100 | 29.0 | 0.078 | 113 |
| 6 | 785 | 130-150 | 0.22 | 228 | 30.4 | 0.027 | 37 |
| 7 | 800 | 150 | | | 2.1 | | 1600 |

FIGS. 1 and 2 show, as an example, the two-photon absorption activity of compounds (3) and (6), respectively. The TPA activity has been characterized by means of “open-aperture” Z-scan measurements of DMSO solutions of the described compounds and with a laser source operating at 780-790 nm with a pulse duration of 130-150 fs.

The Z-scan technique is one of the two most important experimental procedures to measure nonlinear absorption phenomena (two-photon absorption). The open-aperture Z-scan enables the measurement of the nonlinear absorption of the sample by recording the transmittance T (the ratio between transmitted and incident intensity) as a function of the incident intensity. To do this, the sample is moved along the propagation direction (the Z axis) of a focused laser beam. The energy of the laser beam is kept constant, while the intensity grows up as the sample moves towards the focal plane (Z=0). Only the linear transmittance contributes to the signal far from the focal plane. In the proximity of the focus the intensity grows up very quickly and the nonlinear absorption process generates a dip in the transmittance (T<1). The dip is symmetrical with respect to the position of the focal plane. When a fs source is employed, the Z-scan allows for the discrimination between simultaneous TPA and sequential multiphoton absorption processes, involving intermediate excited states populated by nonradiative phenomena. In fact, in the case of fast (100-200 fs) pulses, the latter process does not contribute to the signal, being the nonradiative processes active in the picosecond (ps) or nanosecond (ns) regime. Since the two-photon absorption scales quadratically, and not linearly, with the intensity of the incident laser radiation, the measured absorption as a function of the incident intensity provides unequivocal evidence that the sample is a non-linear (two-photon) absorber. In this way the two-photon absorption parameters β and σ′₂ are experimentally obtained.

FIGS. 1 and 2 show the Z-scan profiles for DMSO solutions of molecules (3) and (6), respectively, measured in a 1-mm cell and with pulse energy of 0.17 and 0.16 mJ. The normalized transmittance (I(z)/I(∞), where I(∞) is the transmitted intensity far from the focal plane) is plotted as a function of the sample position (Z). The deep dip shown in both graphs is a clear evidence that a strong two-photon absorption is occurring in solution. In addition, the Figures ensure that the two compounds show no significant linear absorption at 785 nm and, therefore, are completely transparent at low intensities of the incident radiation (Z far from the focal plane). FIG. 1 proves that compound (3) shows a transmittance T=0.77 at the focal point with a laser pulse energy of 0.17 mJ. This value is remarkably lower, that is the two-photon absorption is larger, than that obtained for other known molecules of comparable molecular weight using the same laser power and pulse width conditions.

In a further aspect of the present invention, in addition to optical limiting activity, said compounds are useful for other applications based on their two-photon absorption activity, such as use as imaging agents in confocal laser scanning fluorescence microscopy via two-photon absorption or excitation.

1-16. (canceled)

17. A compound of formula (I) 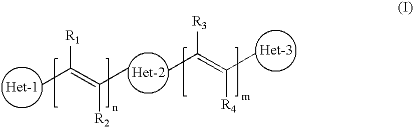
wherein Het-1 and Het-3 are identical or different, and are selected among the following heterocyclic groups: 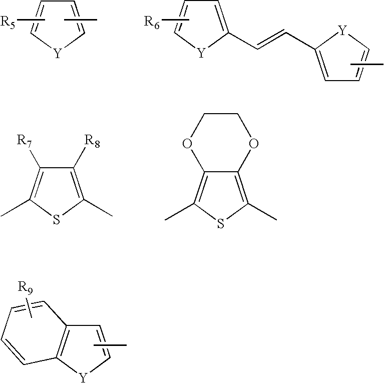
wherein Y may be O, S, or NZ with Z=H, lower alkyl, and aryl; and wherein R₅, R₆, R₇, R₈, and R₉ are the same or different, and are selected from the group consisting of H, alkyl groups having from 1 to 18 carbon atoms, alkoxy, aminoalkyl, alkylhalide, hydroxyalkyl, alkyl groups containing hydroxy and amino functionalities, alkoxyalkyl, alkylsulfide, alkylthiol, alkylazide, alkylcarboxylic, alkylsulfonic, alkylisocyanate, alkylisothiocyanate, alkylalkene, alkylalkyne, aryl, formyl, and that can contain electronpoor ethenylic moieties such as maleimide, capable to react with nucleophilic groups such as —SH, and groups such as isothiocyanate capable to react with groups such as —NH₂;
and Het-2 is selected among the following heterocyclic groups: 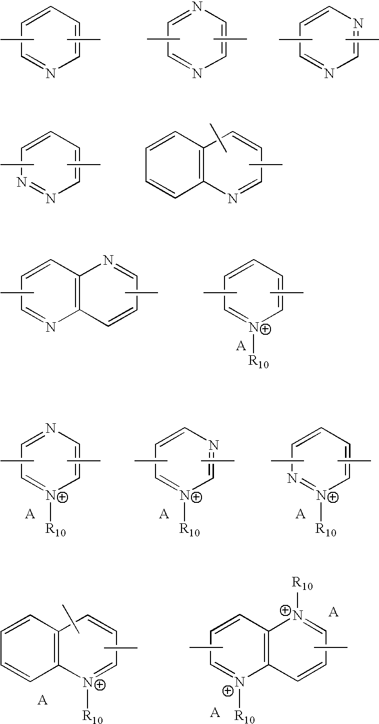
wherein R₁₀ is selected from the group consisting of H, alkyl groups having from 1 to 18 carbon atoms, alkoxy, aminoalkyl, alkylhalide, hydroxyalkyl, alkyl groups containing hydroxy and amino functionalities, alkoxyalkyl, alkylsulfide, alkylthiol, alkylazide, alkylcarboxylic, alkylsulfonic, alkylisocyanate, alkylisothiocyanate, alkylalkene, alkylalkyne, aryl, formyl, and that can contain electronpoor ethenylic moieties such as maleimide, capable to react with nucleophilic groups such as —SH, and groups such as isothiocyanate capable to react with groups such as —NH₂;
and A is selected among the anions alkylsulfonate, arylsulfonate, polyarenesulfonate, triflate, halide, sulfate, methosulfate, phosphate, polyphosphate;
and wherein n and m, the same or different may be 0, 1, 2;
and R₁, R₂, R₃, and R₄, the same or different, may be H, lower alkyl, alkoxyalkyl, aryl, cyano, alkoxycarbonyl, —(CR₁₁R₁₂)ₚ-Het, wherein 0<p<10, R₁₁, and R₁₂, the same or different, are selected from the group of H, lower alkyl, and Het may be Het-1 or Het-2 or Het-3.

18. A compound of formula (II) 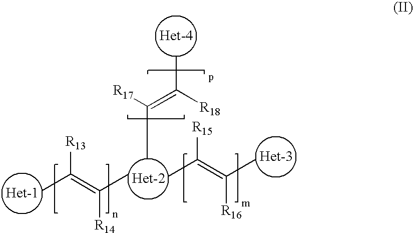
wherein Het-1, Het-3, and Het-4 are the same or different and are selected among the following heterocyclic groups: 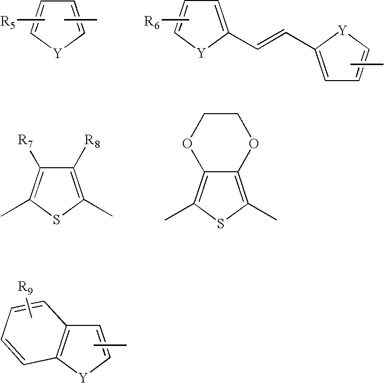
wherein Y may be O, S, and NZ with Z=H, lower alkyl, aryl;
and R₅, and R₆, are the same or different, and are selected from the group consisting of H, alkyl groups having from 1 to 18 carbon atoms, alkoxy, aminoalkyl, alkylhalide, hydroxyalkyl, alkyl groups containing hydroxy and amino functionalities, alkoxyalkyl, alkylsulfide, alkylthiol, alkylazide, alkylcarboxylic, alkylsulfonic, alkylisocyanate, alkylisothiocyanate, alkylalkene, alkylalkyne, aryl, formyl, ketone, and that can contain electronpoor ethenylic moieties such as maleimide, capable to react with nucleophilic groups such as —SH, and groups such as isothiocyanate capable to react with groups such as —NH₂; R₅, and R₆, the same or different, may further be the following heterocyclic group: 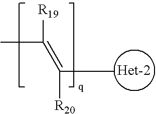
and R₇, R₈, and R₉ are defined as in claim 1;
and Het-2 is defined as in claim 1;
and wherein n, m, p, and q, the same or different, may be 0, 1, or 2;
and wherein R₁₃, R₁₄, R₁₅, R₁₆, R₁₇, R₁₈, R₁₉, and R₂₀ are the same or different and are selected from the group of H, lower alkyl, alkoxyalkyl, aryl, cyano, alkoxycarbonyl, —(CR₂₁R₂₂)ₗ-Het, wherein 0<l<10, and R₂₁ e R₂₂, the same or different, are selected from the group of H, lower alkyl, and Het may be Het-1 or Het-2 or Het-3, or Het-4.

19. The compound according to claim 17, having the following formula (6)

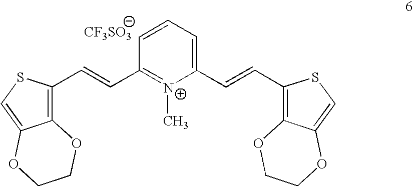

20. The compound according to claim 18, having the following formula (3)

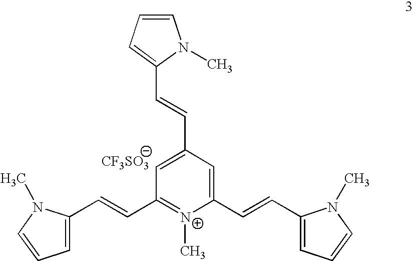

21. The compound according to claim 18, having the following formula (7)

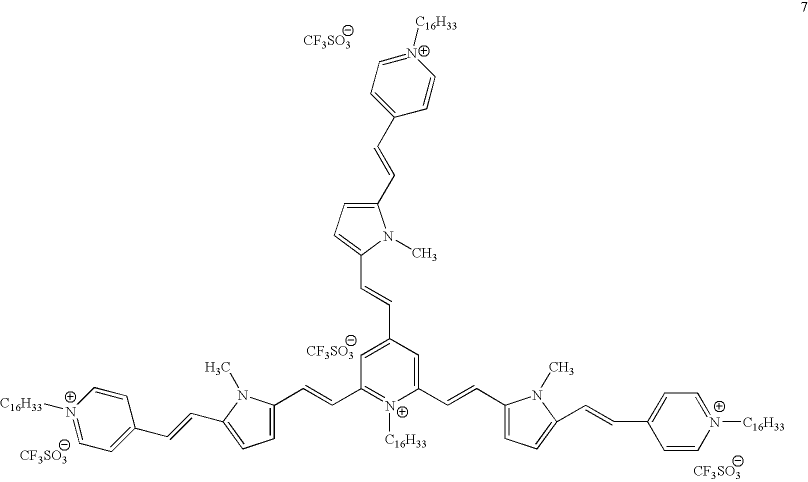

22. A two-photon absorbing chromophore, in solution or in a solid state, being a compound of claim 17.

23. A two-photon absorbing chromophore, in solution or in a solid state, being a compound of claim 18.

24. A compound of general formula (I) according to claim 17 for use in two-photon absorption systems.

25. A compound of general formula (I) according to claim 17 for use as optical power limiting agent via two-photon absorption.

26. A compound of general formula (I) according to claim 17 for use as imaging agent with two-photon absorbing activity for application in detection technologies such as two-photon laser scanning fluorescence microscopy.

27. A compound of general formula (II) according to claim 18 for use in two-photon absorption systems.

28. A compound of general formula (II) according to claim 18 for use as optical power limiting agent via two-photon absorption.

29. A compound of general formula (II) according to claim 18 for use as imaging agent with two-photon absorbing activity for application in detection technologies such as two-photon laser scanning fluorescence microscopy.

30. A composition for use in two-photon absorption systems comprising a compound according to claim 24.

31. A composition for use in two-photon absorption systems comprising a compound according to claim 27.

32. The composition according to claim 30 comprising a polymer material chosen among poly(acrylate), poly(methacrylate), polyimide, polyamic acid, polystyrene, polycarbonate, polyurethane.

33. The composition according to claim 30 comprising an organically-modified silica (SiO₂) network.

34. The composition according to claim 32, wherein said compound is linked to the said polymer material by covalent bonds.

35. The composition according to claim 33, wherein said compound is linked to the said silica network by covalent bonds.

36. The composition according to claim 30 for use as optical power limiting agent via two-photon absorption.

37. The composition according to claim 30 for use as imaging agent with two-photon absorbing activity for application in detection technologies such as two-photon laser scanning fluorescence microscopy.

38. A composition according to claim 31 comprising a polymer material chosen among poly(acrylate), poly(methacrylate), polyimide, polyamic acid, polystyrene, polycarbonate, polyurethane.

39. The composition according to claim 31 comprising an organically-modified silica (SiO₂) network.

40. The composition according to claim 38 wherein said compound is linked to the said polymer material by covalent bonds.

41. The composition according to claim 39 wherein said compound is linked to the said silica network by covalent bonds.

42. The composition according to claim 31 for use as optical power limiting agent via two-photon absorption.

43. The composition according to claim 31 for use as imaging agent with two-photon absorbing activity for application in detection technologies such as two-photon laser scanning fluorescence microscopy.
